(19) United States
(12) Patent Application Publication
(10) Pub. No.: US 2008/0081339 A1
(43) Pub. Date: Apr. 3, 2008

(54) Tumor associated markers in the diagnosis of prostate cancer

(75) Inventors: Brian Liu, Somerville, MA (US); Joshua Ehrlich, Duluth, GA (US); Weiliang Qiu, Brookline, MA (US)

Correspondence Address: LAW OFFICE OF MICHAEL A. SANZO, LLC, 15400 CALHOUN DR., SUITE 125, ROCKVILLE, MD, 20855, US

(73) Assignee: The Brigham and Women's Hospital, Inc., Boston, MA (US)

(21) Appl. No.: 11/905,474

(22) Filed: Oct. 1, 2007

Related U.S. Application Data
(60) Provisional application No. 60/848,637, filed on Oct. 3, 2006.

(51) Int. Cl. C12Q 1/68; G01N 33/53

(52) U.S. Cl. 435/6; 435/7.92

(57) ABSTRACT

The invention is directed to tumor associated markers (TAMs) that can be used diagnostically, especially in the diagnosis of prostate cancer and other markers (BPHMs) that can be used in the diagnosis of benign prostate hyperplasia. It also includes glass or plastic plates or slides on which the TAMs or BPHMs have been immobilized.

### CROSS REFERENCE TO RELATED APPLICATIONS

The present application claims priority to, and the benefit of, U.S. provisional application 60/848,637, filed on Oct. 3, 2006, the contents of which is hereby incorporated by reference in its entirety.

### STATEMENT OF GOVERNMENT FUNDING

The United States Government has a paid-up license in this invention and the right in limited circumstances to require the patent owner to license others under reasonable terms as provided for by the terms of NIH grant U01DK063665, awarded by the Department of Health and Human Services.

### FIELD OF THE INVENTION

The present invention is directed to tumor associated markers (TAMs) that can be used in diagnostics, particularly for prostate cancer. The invention also includes assays in which TAMs are immobilized on plates or slides by monoclonal antibodies and the serum antibody profile of a subject is assessed. In addition, the invention encompasses markers that can be used in the diagnosis of benign prostate hyperplagia.

### BACKGROUND OF THE INVENTION

Prostate cancer is one of the most common malignancies in the United States and, after lung cancer, is the leading cause of cancer-related deaths in men. Currently, the most widely used diagnostic assay for prostate cancer involves measuring the amount of prostate-specific antigen (PSA) in a serum sample. However, this test fails to detect cancer in many men with early stage disease. In addition, there are other prostate-related conditions that can lead to elevated PSA levels and, as a result, only 25-30% of men biopsied for prostate cancer due to an elevated PSA test result are actually found to have the disease. One particularly difficult problem is in distinguishing between men that have benign prostatic hyperplasia (BPH) and cancer. These conditions often produce similar symptoms, including elevated serum PSA levels (Brawer, CA Cancer J. Clin. 49:264-281 (1999)).

A more recent approach to the diagnosis of prostate cancer involves analyzing the antibodies that are generated in a patient when exposed to tumor-specific antigens. Because these antibodies are present in much greater amounts than the antigens that they recognize, the sensitivity of the assays is greatly increased. A number of recent studies have examined the autoimmune response to putative tumor-associated antigens (TAAs) (Liang, et al., J. Clin. Endocrinol. Metab. 71:1666-1668 (1990); Lang, et al., Br. J. Urol. 82:721-726 (1998); Nilsson, et al., Ups. J. Med. Sci. 106:43-9 (2001); Mintz, et al., Nat. Biotechnol. 21:57-63 (2003); Zhou, et al., Biochem. Biophys. Res. Commun. 290:830-838 (2002); Fossa, et al., Cancer Immunol. Immunother. 53:431-8 (2004)). For example, Zheng, et al., demonstrated the presence of serum autoantibodies to a panel of known TAAs in various human cancers, including prostate cancer (Cancer Epidemiol. Biomarkers Prev. 12:136-143 (2003)). Interestingly, cancers of the prostate, breast, and lung showed distinct profiles of antigen-autoantibody reactivity. These results strongly suggest that autoantibody profiling may be a valuable approach for diagnosing cancer and perhaps for determining cancer outcome (Qiu, et al., J. Proteome Res. 3:261-267 (2004); Bouwman, et al., Proteomics 3:2200-2207 (2003); Zhang, Cancer Detect Prev. 28:114-118 (2004); Wang, et al., N. Engl. J. Med. 353:1224-1235 (2005)).

To date, however, nearly all studies of antigen-autoantibody reactivity have relied on arrays of synthetic peptides or recombinant proteins. These arrays may fail to detect the full range of autoimmunity in cancer due to a lack proper post-translational modifications (PTMs) and native protein conformations of the antigens on the array. Several studies have, in fact, shown that PTMs such as phosphorylations (Terzoglou, et al., Clin Exp Immunol. 144:432-439 (2006)), glycosylations (Tramontano, et al., J. Immunol. 172:2367-2373 (2004)) and citrullinations (Vossenaar, et al., Cancer Res. 60:1777-88 (2000)) play a role in stimulating the autoimmune response.

Because of the importance of PTMs in cancer growth and progression, as well as their established link to immunogenicity, we developed a reverse capture autoantibody microarray for use in autoantibody profiling (PCT/US2006/016543; Qin, et al., Proteomics 6:3199-209 (2006); Ehrlich, et al., Nat. Protocols 1:452-60 (2006); Liu, et al., Expert Rev Proteomics 3:283-96 (2006)). The assay has been used to identify a number of markers that can be used either alone or in combination for diagnosing prostate cancer and in distinguishing this disease from benign prostate disease (PCT/US2006/016543). The present application extends upon this previous work and identifies additional diagnostic markers and marker combinations.

### SUMMARY OF THE INVENTION

General Summary

Previous reports have described in detail a microarray assay for examining the antibody profile of a sample of blood, plasma or serum (PCT/US2006/016543; Qin, et al., Proteomics 6:3199-209 (2006); Ehrlich, et al., Nat. Protocols 1:452-60 (2006); Liu, et al., Expert Rev. Proteomics 3:283-96 (2006)). The main characteristic of this assay is that monoclonal antibodies, each recognizing a single known antigen, are bound to a support, such as a glass slide, with each antibody at a separate location. The corresponding antigens are then bound to the immobilized monoclonal antibodies, e.g., by incubating a crude cell lysate with the prepared support. In this way, a microarray is formed in which antigens maintaining their native structural characteristics are immobilized, each antigen at a unique site on the assay support. In the next step, the IgG fraction is isolated from a “test sample” of blood, plasma or serum, i.e., a sample undergoing examination, and the “test antibodies” thus obtained are detectably labeled with a fluorescent dye. These labeled antibodies are then combined with an equal amount of “control antibodies” that have been isolated from a second sample of blood, serum or plasma (e.g., from a subject known to be disease free). The control antibodies are attached to a second fluorescent label that is different from and distinguishable from the label used for the test antibodies. The mixture of labeled test and control antibodies is incubated with the immobilized antigens and the relative amount of binding is determined based upon the detectable labels. The assay procedure can be used to compare the antibodies present in patients having a disease such as cancer to the antibodies in samples from normal individuals. Results have indicated that the procedure can be used to identify antigens that are characteristic of prostate cancer, ovarian cancer and progressive benign prostate hyperplasia (see PCT/US2006/016543).

Specific assays performed using antibodies derived from patients with prostate cancer and from patients with benign prostate hyperplasia are described in detail in the Examples section below. Based upon these assays, 28 antigens were identified that are characteristic of prostate cancer and that may be used diagnose this disease. These are shown in Table 3 along with an accession number for the Swiss Protein database (a compilation of protein sequences well known in the art). Each accession number is associated with a unique amino acid sequence that unambiguously defines the protein and which is readily accessible to the public. In addition, Table 3 provides abbreviations for the antigens that we will use herein for convenience. It was calculated that a multiplexed platform consisting of 5 selected antigens could be used to detect prostate cancer in a high percentage of subjects.

The 28 antigens shown in Table 3 may be grouped into three categories. First, there are antigens that have previously been identified as useful in diagnosing prostate cancer using antibody profiling assays similar to those described herein (see PCT/US2006/016543). These include: CHD-3 (Swiss protein accession no. Q12873); NFAT (Q13469); CALD1 (Q05682); p53 (P04637); SP11 (P17947); EPHX1 (P07099); DGKq (P52824); TP73 (015350); and CSE1L (P55060).

The second category includes antigens that have not been previously identified using antibody profiling but which have been reported as having characteristics that might suggest a role in prostate cancer. These are EGFR (P00533); AR (P10275); (CCND1); and CASP-8 (Q14790).

Finally, there are antigens that appear to have not previously been associated with prostate cancer at all. These include: SATB1 (Q1826); PEX1 (O43933); CRP2 (P52943); PSME3 (Q12920); GFAP (P14136); STX6 (O43752); SOS1 (Q07889); HSF4 (Q9ULV5); SRP54 (P13624); NHE-3 P19634); PKP2 (Q99960); GRIN2B (Q13224); GSPT2 (P06493); STAT2 (P52630); STIM1 (Q13586).

In addition to the 28 TAMs described above, 52 antigens have been identified that are characteristic of benign prostate hyperplagia. These are shown in Table 4.

Detailed Summary

In its first aspect, the invention is directed to a method of diagnostically evaluating a subject for prostate cancer by obtaining a “test” biological sample and assaying the sample for one or more of the following tumor associated markers (TAMs): SATB1 (Q1826); PEX1 (O43933); CRP2 (P52943); PSME3 (Q12920); GFAP (P14136); STX6 (O43752); SOS1 (Q07889); HSF4 (Q9ULV5); SRP54 (P13624); NHE-3 (P19634); PKP2 (Q99960); GRIN2B (Q13224); GSPT2 (P06493); STAT2 (P52630); and STIM1 (Q13586). The results from the test biological sample are compared to those from one or more similar “control samples” obtained from subjects known to be disease free or to have benign prostate disease, e.g., benign prostate hyperplasia. If the comparison indicates that the test sample has a higher amount of one or more TAMs, this is an indication that the test subject has prostate cancer. As the number of elevated TAMs increases, so does the probability that prostate cancer is present.

Examples of test biological samples that can be used include blood, plasma, serum, urine, prostate tissue and prostate fluid (i.e., fluid immediately surrounding the prostate gland). The most preferred of these is blood, plasma or serum. The amount of TAM present in the biological sample can be determined by any method known in the art, e.g. by ELISA, radioimmunoassay or radioreceptor assay. The most preferred method however is by an antibody profiling assay. For the purposes of the present application, this is defined as assessing the amount TAM present indirectly by examining the amount of antibody against the TAM in the biological sample. Specific examples are provided herein and in described herein and in PCT/US2006/016543.

In addition to assaying samples for one or more of the TAMs described above, samples may also be assayed for at least one additional TAM indicative of prostate cancer and selected from: CHD-3 (Swiss protein accession no. Q12873); NFAT (Q13469); CALD1 (Q05682); p53 (P04637); SP11 (P17947); EPHX1 (P07099); DGKq (P52824); TP73 (015350); CSE1L (P55060); EGFR (P00533); AR (P10275); CCND1 (P24385); and CASP-8 (Q14790). In all cases, it is preferred that samples be assayed for at least 5 of the cancer specific TAMs, more preferably at least 10 and still more preferably for all 28. In addition, assays may include a determination of the amount of prostate specific antigen (PSA) present.

In another aspect, the invention includes a glass or plastic plate or slide having at least 5 different TAMs, each attached at a different position. The 5 most preferred antigens are: NFAT1 (Q13469), HSF4 (Q9ULV5), p53 (P04637), CASP8 (Q14790), and SP11 (P17947). At least one of the TAMs must be selected from: SATB1 (Q1826); PEX1 (O43933); CRP2 (P52943); PSME3 (Q12920); GFAP (P14136); STX6 (O43752); SOS1 (Q07889); HSF4 (Q9ULV5); SRP54 (P13624); NHE-3 (P19634); PKP2 (Q99960); GRIN2B (Q13224); GSPT2 (P06493); STAT2 (P52630); and STIM1 (Q13586). The other TAMs are selected from: SATB1 (Q1826); PEX1 (O43933); CRP2 (P52943); PSME3 (Q12920); GFAP (P14136); STX6 (O43752); SOS1 (Q07889); HSF4 (Q9ULV5); SRP54 (P13624); NHE-3 P19634); PKP2 (Q99960); GRIN2B (Q13224); GSPT2 (P06493); STAT2 (P52630); STIM1 (Q13586); CHD-3 (Swiss protein accession no. Q12873); NFAT (Q13469); CALD1 (Q05682); p53 (P04637); SP11 (P17947); EPHX1 (P07099); DGKq (P52824); TP73 (015350); CSE1L (P55060); EGFR (P00533); AR (P10275); (CCND1); and CASP-8 (Q14790). Preferably each TAM is attached to the plate or slide by a monoclonal antibody that specifically recognizes it. In a preferred embodiment at least 10 TAMs are attached to the plate or slide and in the most preferred embodiment all 28 TAMs are attached. In addition, PSA may optionally be attached.

The plate or slide with attached TAMs may be included as part of a kit along with instructions concerning its use in performing a diagnostic assay for prostate cancer. Optionally, the kit may also include a control sample derived from one or more individuals known not to have prostate disease or from one or more patients with benign prostate hyperplasia.

The invention also includes an assay for comparing the antibodies present in samples of blood, plasma or serum. The assay involves obtaining an immobilized array of TAMs, each TAM being attached to the surface of a solid support by an antibody that specifically recognizes it. The TAMs are selected from: EGFR (P00533); AR (P10275); (CCND1); and CASP-8 (Q14790); SATB1 (Q1826); PEX1 (O43933); CRP2 (P52943); PSME3 (Q12920); GFAP (P14136); STX6 (O43752); SOS1 (Qo7889); HSF4 (Q9ULV5); SRP54 (P13624); NHE-3 P19634); PKP2 (Q99960); GRIN2B (Q13224); GSPT2 (P06493); STAT2 (P52630); STIM1 (Q13586). Test antibodies are then derived from a first sample of blood, serum or plasma and attached to a first detectable label. Control antibodies derived from a second sample of blood, serum or plasma are also obtained and are attached to a second detectable label that can be distinguished from the first detectable label after incubation with the immobilized TAMs. In the next step, the labeled test antibodies and labeled control antibodies are incubated with the array of immobilized TAMs. Unbound labeled antibodies are then removed and the amount of the first and second detectable labels associated with each TAM is determined.

In a preferred embodiment, the first and said second detectable labels are dyes or fluorescent labels chosen so that the first detectable label absorbs or fluoresces at a different wavelength than the second detectable label, e.g., Cy3 and Cy5 fluorescent dyes may be used. Preferably, the test antibodies are from a subject that is known to have a specific disease or condition, e.g., cancer, and the control antibodies are from a subject that does not have the disease or condition. Using this procedure, TAMs specific for a disease or condition and of potential diagnostic value may be identified.

In addition, to the 28 TAMs associated with prostate cancer, 52 marker antigens that are characteristic of benign prostate hyperplasia (BPH) were identified and are shown in Table 4. Increased expression of these markers in a patient's serum relative to expression in control serum derived from disease free individuals is an indication of the presence of BPH. Preferably at least 5 of the BPH markers are examined in making a diagnosis. As with the TAMs discussed above, the BPH markers may be attached to a plate or slide (preferably by means of a monoclonal antibody) and used in assays. Between 5 and 52 different markers should be present on the plates or slides which may also include some, or all, of the TAMs described above and also PSA. The plates or slides may be included as part of a kit along with instructions for using the plates or slides in assays for BPH.

### BRIEF DESCRIPTION OF THE DRAWINGS

FIG. 1: FIG. 1 represents a schematic of a “reverse capture” microarray. Well-characterized, highly specific, and high affinity monoclonal antibodies are spotted on an array surface. Cell extracts containing the antigens are then immobilized to the respective spotted antibodies. This is then followed by incubation with labeled autoantibodies from a patient's serum. Test and control autoantibodies are then labeled with different CyDyes, and the ratio of the fluors determines the relative abundance of the autoantibodies in a given serum sample.

### DETAILED DESCRIPTION OF THE INVENTION

The present invention is based upon the identification of 28 antigens that can be used to identify patients with prostate cancer. These are shown in Table 3 and are all well known in the art. Although an increase in any of these in the serum of a subject is suggestive of the presence of prostate cancer, a much better assessment can be made by examining many, preferably all of the antigens. One way of doing this is to use an ELISA, radioimmuno- or radioreceptor assay to examine individual antigens.

Alternatively, microarray plates can be used to examine multiple antigens at once. The most preferred method of doing this is to immobilize an array of monoclonal antibodies, each recognizing a specific antigen, to an inert surface. Many plastic, glass or nylon surfaces are known in the art and can be used for this purpose. Monoclonal antibodies appropriate for attachment are commercially available, e.g., from Clontech Inc. and other manufacturers, and in some cases it may be possible to purchase arrays already attached to a surface. If desired, fragments derived from the monoclonal antibodies that maintain the ability to specifically recognize antigen may also be used.

The next step in the procedure is to attach the antigens to the immobilized antibodies. This may be accomplished by lysing cells derived from culture or in vivo, removing cellular debris and then incubating the crude antigen solution with the array of immobilized antibodies. At the end of the incubation, unattached materials and antigens are removed, thereby leaving behind an array of antigens attached to slides or plates by the immobilized monoclonal antibodies. The identity of each of the attached antigens is known from the specificity of the antibody to which it is attached. In other words, each antibody is at a specific location on the slide or plate and recognizes only one particular type of antigen.

Once the array of immobilized antigens has been prepared, the next step is to prepare the antibody samples that will undergo testing. A sample of serum, plasma or blood is removed from a test subject being tested for prostate cancer. A second “control” sample of blood, plasma or serum is then obtained from one or more other individuals that do not have the disease or that have an alternative condition, e.g., benign prostate hyperplasia. The IgG fraction present in the samples is then isolated using any method known in the art and the resulting antibodies are labeled. Any type of label that can be detected using a microarray assay is compatible with the present invention, with fluorescent dyes such as Cy3 and Cy5 being preferred. The main requirement for labeling is that the label attached to the antibodies derived from the test subject must be distinguishable from those derived from the control subject after binding has occurred. Thus, the absorption or emission wave lengths of the dyes should be sufficiently different to allow them to be readily distinguished.

After test and control antibodies have been labeled, an equal amount of each (e.g., 100 μg) is placed in a buffer solution and incubated with the array of immobilized antigens. The incubation buffer may consist of any type of standard buffer used in handling antibodies, e.g., PBS. The incubations may be carried out at about room temperature for a period ranging from 15 minutes to 2 hours with about 45 minutes being generally preferred. At the end of this time, unbound labeled antibody is removed and plates or slides are then analyzed to determine the amount of fluorescence or light absorption associated with each immobilized antigen. By comparing the results obtained using wavelengths characteristic of the dye attached to the test antibodies with those characteristic of the dye attached to the control antibodies, a profile can be obtained in which antibodies preferentially present in the test sample are identified. The presence of such antibodies is an indication that the antigens that they recognize are produced to a greater extent in the test subject.

Microarray plates or slides containing an array of the 28 TAMs (or a subset of the TAMs) may be prepared and included as part of a kit. The kit will also include instructions describing how the plates or slides can be used in diagnostic assays for prostate cancer. In addition, it may include other components needed in assays such as buffers or a “control” preparation of antibodies.

Although the antigens that have been identified herein are characteristic of prostate cancer, it is expected that some of the antigens, or combinations of antigens will also be useful in diagnosing other types of cancer as well. Included among these are cancers of the ovary, breast, colon, lung, stomach, pancreas, liver, kidney esophagus, and brain. Assays utilizing arrays of the TAMs in Table 3 may also be combined with assays of other factors of diagnostic value. For example, assays of prostate specific antigen may be used to provide further information relevant to a diagnosis of prostate cancer.

### EXAMPLES

In the current study, we examined differential autoantibody expression between prostate cancer and benign prostatic hyperplasia (BPH) patients to native prostate tumor antigens. The platform used in this research was the reverse capture autoantibody microarray, which we recently developed and described (Qin, et al., Proteomics 6:3199-209 (2006); Ehrlich, et al., Nat. Protocols 1:452-60 (2006)) As a proof-of-concept, a series of 10 reverse capture experiments were carried out and analyzed to test the hypothesis that serum autoantibody profiling can be used to distinguish between prostate cancer and BPH patients with similar blood PSA levels.

Using a reverse capture autoantibody microarray, 28 unique antigens were identified that are differentially targeted by autoantibodies in patients with prostate cancer, as compared to patients with BPH. Cross-validations were performed, and sample identity—prostate cancer or BPH—was correctly predicted in 82.34-89.56% of cases.

Materials and Methods

Cell Culture and Lysis

All native antigens for the reverse capture experiments were extracted from established human prostate tumor cell lines. LNCaP (androgen-responsive) and PC-3 (androgen-independent) cells were obtained from the American Type Culture Collection (Rockville, Md.). Cells were cultured in RPMI with L-glutamine (Invitrogen Corp., Carlsbad, Calif.), supplemented with 10% FBS and 100 IU/mL penicillin and 100 ug/mL streptomycin. Whole cell extracts were obtained by scraping cells from plates and resolving the cell pellets in Protein Extraction/Labeling Buffer (Clontech, Mountain View, Calif.). After rotating the suspension for 10 min at room temperature, the insoluble fraction was removed by centrifugation (30 min at 10 000×g at 4° C.). The protein concentration in the lysates was determined using a BCA™ Protein Assay Reagent kit according to the manufacturer's instructions (Pierce Biotechnology, Rockford, Ill.).

Serum Collection and IgG Purification

Serum samples were obtained from BPH patients during routine clinical visits and from patients with prostate cancer during pre- and post-operative evaluations. Serum samples were aliquoted and stored at −80° C. until use. To establish a control group, we screened the BPH serum bank and selected five patients with histologically diagnosed BPH; each was followed for a minimum of two years (mean follow-up time=5.6 years) to rule-out a diagnosis of cancer. IgGs were isolated from the sera of each BPH patient and pooled together in equal quantities. 10 biopsy-positive patients with prostate carcinoma were matched to the BPH control group for age and pre-operative blood PSA level. IgG was separately purified and aliquoted from each of the pre-operative serum samples of these patients. Tables 1 & 2 show the relevant clinical characteristics of the BPH and prostate cancer patient cohorts. Also, IgG from 9 of 10 cancer patients was purified from sera drawn ≧1 year (mean=1.7 years) post-surgery; these IgGs were pooled in equal quantities. All IgG purifications were performed using the Melon™ Gel IgG Purification Kit (Pierce Biotechnology, Rockford, Ill.) according to the manufacturer's instructions. Eluted IgG samples were re-purified using fresh reagents to ensure the exclusive isolation of IgG from patient sera. IgGs were adjusted to a concentration of 1 mg/mL using the Melon™ Gel Purification Buffer (contained in kit) and stored at −20° C. until use. The purity of the IgGs was determined by running aliquots onto 8% SDS-PAGE gels.

Differential Labeling of IgGs with Fluorescent Dyes

Purified IgGs were labeled with DyLight™ 547 and 647 fluorophores (Pierce Biotechnology, Rockford, Ill.). For each experiment, 100 ug of prostate cancer test IgG and 100 ug of BPH control IgG was labeled with each of the two dyes, DyLight™ 547 and DyLight™ 647. Unbound dye was removed using the Zebra™ Desalt Spin Columns contained in the dye kits (all described in detail in our published protocol) (Ehrlich, et al., Nat. Protocols 1:452-60 (2006)).

Antibody Microarray

The antibody microarray used in the reverse capture experiments was the Clontech™ Ab Microarray 500, consisting of 500 unique, well-characterized monoclonal antibodies (mAbs) spotted in duplicate on a glass slide. The manufacturer tests all 1000 arrayed antibodies for proper orientation, specificity and sensitivity. A variety of cytosolic and membrane target antigens are represented by the mAbs on the array. Targets include proteins involved in cell-cycle regulation, gene transcription and translation, signal transduction, apoptosis, cell growth and oncogenesis. For a complete list of target antigens, including SwissProt ID numbers, see: http://bioinfo.clontech.com/abinfo/array-list-action.do.

The Reverse Capture Platform

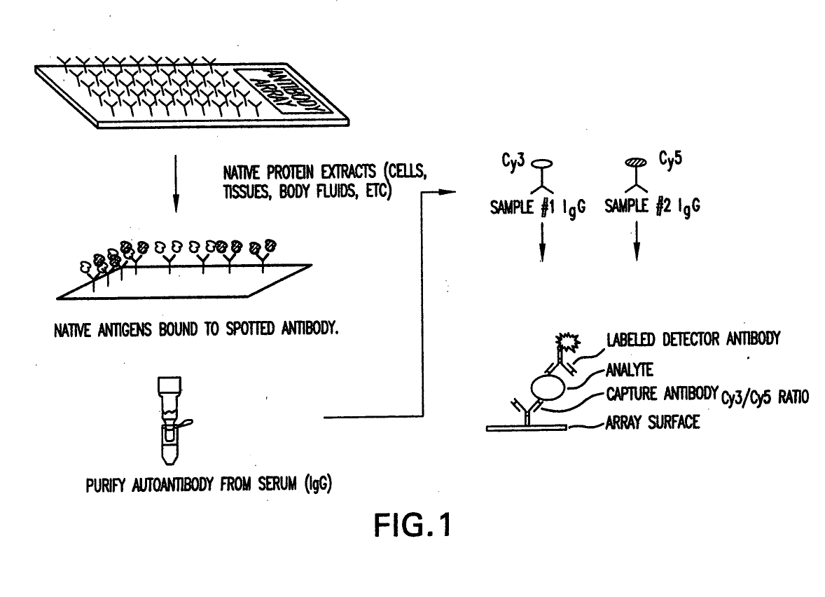

All reverse capture experiments were carried out as previously described (see FIG. 1; Ehrlich, et al., Nat. Protocols 1:452-60 (2006)). Briefly, native antigens were acquired from mixed PC-3 and LNCaP cell extracts and were incubated with each microarray slide. With native tumor antigens immobilized on the microarray, differentially labeled test and control IgG samples were incubated with each slide, according to the dye-swap method. The arrays were then washed, and the slides were dried by centrifugation.

Scanning and Quantitation of Reverse Capture Data

Slides were scanned on the PerkinElmer ScanArray 4000XL scanner. The four images corresponding to each of the two labeled IgG samples incubated with each array slide were saved as single-file TIFF images. Data was extracted from these images by overlaying the corresponding GAL files found on the Clontech website (http://bioinfo.clontech.com/abinfo/array-list-action.do) using the GenePix Pro 6.0 software (Molecular Devices, Sunnyvale, Calif.). Data points were quantified and results were saved as GenePix Results (GPR) files.

Statistical Methods

For each individual slide, duplicate intensities for target antigens were averaged and the median foreground intensity level was used for analysis. Controls, antigen targets with at least one intensity reading less than then minimum intensity of the negative controls, and antigens with one or more non-zero flags were removed from the analysis. For each reverse capture experiment two ratios were obtained:
Mix 1 ratio: BPH (DyLight™ 647)/Cancer (DyLight™ 547)
Mix 2 ratio: Cancer (DyLight™ 647)/BPH (DyLight™ 547)

We performed a log transformation of these ratios and, using ratio-based normalization, centered and scaled the two sets of log ratios for each experiment across antigen targets so the range of each log ratio data set was equal. Next, a Student's t-test was performed on each antigen target, comparing data taken from the two different mixes across the set of 10 experiments. The significant level of differential expression for autoantibodies to a given target was set at p≦0.01. The power of this analysis was then calculated using the method of Lee and colleagues (Lee, et al., Stat Med. 21:3543-3570 (2002)).

Cross-Validation/Predictive Modeling

The 20 reverse capture array slides were randomly partitioned into 5 groups (4 slides per group). 4 of these groups (16 slides) were designated as a training set and 1 as a test set (4 slides). A list of significant autoantigens was generated (p≦0.01) based on the training set. The identity, prostate cancer or BPH, of each test sample was then predicted using the Random Forest (Breiman, L., Machine Learning 45:5-32 (2001)) and k-nearest neighbor (Ripley, B D. Pattern Recognition and Neural Networks, Cambridge University Press, 1996; k=5) decision tree algorithms. Five-fold cross-validation was performed and reiterated 500 times. In each case a new list of significant autoantigens was generated based on the specific array data comprising the training set, and the identity of test samples was predicted. A prediction error rate was calculated by denoting Mk as the number of prediction errors when the k-th part is regarded as the test set. An estimate of the prediction error rate based on 5-fold cross-validation is e=(m₁+m₂+m₃+m₄+m₅)/40.

Results

Differential Autoantibody Reactivity

We performed a series of 10 reverse capture experiments in order to identify autoantibody biomarkers of prostate carcinoma. In each of these experiments, the autoantibody profile of a different prostate cancer patient was compared to a constant group of five BPH control patients (see Tables 1, 2). Test and control groups were matched for age and blood PSA level in order to eliminate variation of known parameters. It was observed that, overall, individual cancer patients exhibit similar autoantibody profiles to one another, but that these patients' profiles were distinct from the BPH autoantibody profile.

Autoantibody Profiling

In order to analyze the data from the set of 10 reverse capture experiments, the median foreground intensity level for each antigen was considered. Since every experiment was performed with a two-slide dye-swap, two fluorescence intensity ratios were obtained from each experiment and centering and scaling of these ratios was carried out to eliminate dye effects and variation between experiments.

When the log-transformed, normalized data were analyzed, we found 28 unique antigens with p-values ≦0.01 that were differentially targeted by autoantibodies from patients with prostate cancer. These antigens are listed in Table 3. We then calculated the power of our results using the method of Lee and colleagues. This method reported that we should expect to discover 95.02% of significant antigen-autoantibody reactivities when the mean number of false positives permitted is set at only 1.

Finally, the log ratios of the fluorescence intensities of the significant prostate cancer and BPH autoantigens were used to perform 2-D hierarchical clustering and to construct a heat map using the Euclidean similarity metric (Gibbons, et al., Genome Res. 12:1574-81 (2002)). This metric is used to transform data points into clusters that use relative distances to reflect similarities between substances. The antigens listed in Table 3 are the same prostate cancer autoantibody target antigens found on the heat map.

Post-Operative Profiling

A reverse capture experiment was performed to examine the autoantibody repertoire of prostate cancer patients after radical prostatectomy. All of the 9 original cancer patients who remained in the study experienced a positive surgical outcome, as defined by a sustained drop in blood PSA below 0.01 ng/mL (mean follow-up=1.7 years). Since differential profiling was determined based on relative fluorescence levels, it was useful to maintain the identical BPH control used in the initial set of experiments; without this control present on each array slide, the basis for comparing pre-operative and post-operative relative fluorescence levels would be lost. Pooled test IgG from serum drawn from prostate cancer patients at least 1 year post-surgery was used in this experiment, and a one-sample t-test was performed on the normalized, dye-swapped data to determine significant deviation (p≦0.01) from the expected mean log ratio of 0.0. It was observed that only one of the previously identified 28 prostate cancer autoantigens, SRP54, remained differentially targeted by autoantibodies in post-surgery prostate cancer patients.

Autoantibodies as Biomarkers

Since each of the ten experiments in this study was performed using the two-slide dye-swap method, a total of 20 array slides were evaluated for relative autoantibody expression levels. For the 28 identified prostate cancer target antigens, on average, prostate cancer autoantibody expression exceeded BPH expression on 72.14% (+/−7.38%) of reverse capture slides. We carefully selected 5 of the 28 significant antigens and averaged the percent of array slides on which each was differentially targeted by prostate cancer autoantibodies. These 5 antigens were: NFAT1 (exceeded in 90% of the slides), HSF4 (exceeded in 85% of the slides), p53, CASP8, and SP11 (exceeded in 80% of the slides respectively). The result of this computation implies that with a platform consisting of 5 autoantigens it may be possible to detect prostate carcinoma at a rate of up to 83%.

In order to cross-validate the results of this study, we developed a classification model. The data from the 20 reverse capture slides were randomly partitioned into five groups, each consisting of 4 array slides. Four-fifths of the data (16 array slides) was designated as a training set and the remaining one-fifth was used as a test set. Two different tree-based decision algorithms, Random Forest and k-nearest neighbor (k=5), were used to classify each sample in the test set as either prostate cancer or BPH based on its similarity to the data contained in the training set. One of these methods, Random Forest, is a classification system that uses many unique decision trees and gives as its output the mode of all of the individual trees (Breiman, L., Machine Learning 45:5-32 (2001)). The other method, k-nearest neighbor, identifies expression patterns that are uniformly high in one class and uniformly low in others, and, using decision trees, correlates the test sample with its closest k neighbors (Ripley, B D. Pattern Recognition and Neural Networks, Cambridge: Cambridge University Press, 1996)). Both of these methods predict the identity of designated test samples based on the known identities and patterns of the training samples.

For both methods of analysis, five-fold cross validation was performed on 500 unique partitionings of the 20 array slides. In each reiteration of partitioning, a different combination of array slides was randomly assigned to the training and test sets. A list of significant autoantigens (p≦0.01) was generated for every unique training set. Both of the prediction models employed yielded similar results. The Random Forest method had a prediction error rate of 13.56% (+/−3.12%), while the 5-nearest neighbor method had a prediction error rate of 14.34% (+/−3.32%). Thus, in 82.34-89.56% of cases, samples were properly classified as either prostate cancer or BPH.

### DISCUSSION

In this study, we employed a reverse capture autoantibody microarray to determine the specific antigen-autoantibody reactivities which best characterize the body's immune response to prostate cancer and that may be used to screen for, diagnose, and/or treat the disease. A series of 10 reverse capture experiments on 20 microarray slides was used to identify 28 unique antigens that were differentially targeted by autoantibodies from patients with prostate carcinoma (see Table 3).

The 28 prostate cancer autoantigens that we identified perform a variety of intracellular functions that can be placed into five broad categories: i) apoptosis; ii) cell cycle regulation; iii) transcription factors; iv) kinases; and, v) cancer growth factors and receptors. Several of these antigens, including, epidermal growth factor receptor (EGFR; Kim, et al, Curr. Opin. Oncol. 13:506-13 (2001)), tumor suppressor protein p53 (Lasky, et al., Environ. Health Perspect. 104:1324-1331 (1996)), and tumor suppressor protein p73 (Tominaga, et al., Br. J. Cancer. 84:57-63 (2001)) have reported associations with various types of cancer. Another autoantigen identified in this study, androgen receptor (AR), has been widely cited in association with malignant prostate disease (Linja, et al., J. Steroid Biochem. Mol. Biol. 92:255-64 (2004)). Additionally, a number of the target antigens that we identified have no previous association with prostate cancer, and their role in the disease process should be further investigated.

One prostate cancer autoantigen that was identified, Cyclin D1 (CCND1), is an oncogene that is reportedly upregulated in a number of neoplastic diseases (Bates, et al., Oncogene 9:71-79 (1994)). This protein is involved in the regulation of the G1/S phase transition of the cell cycle, and its expression is believed to be dependent upon the cell cycle itself. Aaltomaa and colleagues demonstrated that CCND1 expression levels in human prostate tissue are related to a number of malignant cellular features (Aaltomaa, et al., Prostate 38:175-182 (1999)). They showed that CCND1 expression is related to tumor node metastasis status, histological differentiation, perineural invasion, DNA ploidy, S-phase fraction and mitotic index. Furthermore, their study demonstrated the ability to predict cancer-related survival based on CCND1 levels in prostate tissue.

Another antigen that we found to be differentially targeted by prostate cancer patient autoantibodies was Caspase 8 (CASP-8). The caspases are a family of cysteine proteases involved in an important apoptotic signaling pathway (Nunez, et al., Oncogene 17:3237-3245 (1998)). Defects in the regulation and/or expression of the caspases are reportedly involved in a variety of diseases, including, neurodegenerative disorders, autoimmune diseases and cancer (Ho, et al., FEBS J. 272:5436-5453 (2005)). Using Western blotting, Vincent and colleagues demonstrated that two different CASP-8 isoforms were upregulated in four distinct prostate tumor cell lines as compared to two normal prostate cell lines (Vincent, Prostate 66:987-995 (2006)).

Our ability to identify CCND1, CASP-8 and other antigens of interest as TAAs in prostate cancer suggests that the reverse capture platform is a highly effective tool for autoantibody profiling, and that its results may make a valuable contribution to the development of new biomarkers and the identification of novel therapeutic targets.

We cross-validated the data from this study by testing its ability to correctly classify randomly designated test samples as prostate cancer or BPH. The method that was used to partition data and construct the training and test sets is highly robust and contributes to the significance of our results. The partitioning of data was done by randomly assigning array slides to either the training or test group. Since 4,845 unique combinations are possible when of selecting 4 array slides out of 20 (20!/4!(20-4)!), we chose to reiterate the partitioning of training and test data a total of 500 times, while performing 5-fold cross-validation with each partitioning of the slides. It is important to note that a list of significant antigen-autoantibody reactivities was generated separately for each of the training sets, and that the specific corresponding list was used as the basis for classification of the samples represented on the 4 slides in each test group of arrays.

The results given by the two classification algorithms, Random Forest and k-nearest neighbor, are similar and equally suggest that autoantibody production to the antigens that we identified may serve as a highly sensitive and specific marker of prostate carcinoma. The error prediction rates of the two methods range from 10.44-17.66%. Additionally, the high power achieved by our data, 95.02% detection of differential expression with only one false positive permitted, confirms the existence of significant differences between prostate cancer and BPH that may be used to classify unknown samples. These results suggest that using the reverse capture platform to evaluate autoantibody expression, it may be possible to correctly classify up to 89.56% of patient samples as prostate cancer or BPH with a very low false-positive rate.

The autoantibody repertoire observed among post-operative cancer patients further validates these results of this study. We found that following radical prostatectomy the autoantibody profile of patients with prostate cancer changes significantly. After undergoing surgery, only one of the 28 prostate cancer-specific autoantigens remained differentially targeted by the same patients whose autoantibodies overwhelmingly targeted all of these TAAs prior to surgery. This result implies that nearly all of the identified prostate cancer antigen-autoantibody reactivities may be significant to the disease process and useful as markers of its presence.

TABLE 1
Benign prostatic hyperplasia patient clinical data

| Sample number | Age | PSA | Length of |
|---|---|---|---|
| (internal code) | (years) | (ng/mL) | follow-up (years) |
| 1 (BPH42-3) | 71 | 9.5 | 9 |
| 2 (BPH47-1) | 68 | 4.7 | 9 |
| 3 (BPH121-1) | 57 | 4.0 | 2 |
| 4 (BPH122-1) | 51 | 5.2 | 6 |
| 5 (BPH125-1) | 75 | 6.1 | 2 |
| Mean | 64.4 | 5.90 | 5.6 |

TABLE 2
Prostate cancer patient clinical data

| Sample number | | PSA | | |
|---|---|---|---|---|
| (internal code) | Age (years) | (ng/mL) | Gleason grade | TNM stage |
| 1 (PC41) | 59 | 7.9 | 3 + 4 | T2b |
| 2 (PC47)* | 67 | 5.2 | 3 + 4 | T2c |
| 3 (PC50) | 58 | 5.1 | 3 + 3 | T2a |
| 4 (PC56) | 63 | 6.3 | 3 + 3 | T2a |
| 5 (PC74) | 62 | 5.4 | 3 + 3 | T2a |
| 6 (PC76) | 69 | 7.9 | 4 + 5 | T3a |
| 7 (PC79) | 59 | 4.9 | 3 + 4 | T3a |
| 8 (PC87) | 63 | 5.0 | 3 + 3 | T2c |
| 9 (PC93) | 61 | 5.6 | 3 + 3 | T2a |
| 10 (PC115) | 62 | 5.3 | 3 + 4 | T2c |
| Mean | 62.3 | 5.86 | n/a | n/a |

PSA: prostate specific antigen
TNM stage: tumor node metastasis staging system
*withdrew from study prior to 1 year follow-up

TABLE 3
Differentially targeted autoantigens in prostate cancer

| Antigen | Swiss-Prot | | |
|---|---|---|---|
| abbreviation | accession no. | Description | p-value |
| CHD-3 | Q12873 | Chromodomain helicase-DNA-binding protein 3 | 0.0001 |
| NFAT1 | Q13469 | Nuclear factor of activated T-cells, cytoplasmic 2 | 0.0001 |
| EGFR | P00533 | Epidermal growth factor receptor | 0.0004 |
| SATB1 | Q01826 | Special AT-rich sequence-binding protein 1 | 0.0006 |
| PEX1 | O43933 | Peroxisome biogenesis factor 1 | 0.0008 |
| CRP2 | P52943 | Cysteine-rich protein 2 | 0.0008 |
| CALD1 | Q05682 | Caldesmon 1 | 0.0009 |
| AR | P10275 | Androgen receptor | 0.0010 |
| p53 | P04637 | Tumor protein p53 | 0.0010 |
| PSME3 | Q12920 | Proteasome activator complex subunit 3 | 0.0012 |
| CCND1 | P24385 | Cyclin D1 (PRAD1) | 0.0027 |
| GFAP | P14136 | Glial fibrillary acidic protein, astrocyte | 0.0028 |
| STX6 | O43752 | Syntaxin 6 | 0.0038 |
| SOS1 | Q07889 | Son of sevenless homolog 1 | 0.0039 |
| CASP-8 | Q14790 | Caspase 8 | 0.0040 |
| SPI1 | P17947 | Spleen focus forming virus oncogene | 0.0041 |
| HSF4 | Q9ULV5 | Heat shock transcription factor 4 | 0.0042 |
| SRP54 | P13624 | Signal recognition particle 54 kDa protein | 0.0044 |
| EPHX1 | P07099 | Epoxide hydrolase 1, microsomal | 0.0050 |
| DGKq | P52824 | Diacylglycerol kinase (PKC), theta | 0.0055 |
| NHE-3 | P19634 | Solute carrier family 9 (sodium/hydrogen | 0.0055 |
| | | exchanger), isoform 3 regulatory factor 1 | |
| PKP2 | Q99960 | Plakophilin 2 | 0.0055 |
| TP73 | O15350 | Tumor protein p73-like | 0.0058 |
| GRIN2B | Q13224 | Glutamate [NMDA] receptor subunit epsilon 2 | 0.0068 |
| GSPT2 | P06493 | Cell division cycle 2, G1 to S and G2 to M | 0.0069 |
| STAT2 | P52630 | Signal transducer and activator of transcription 2 | 0.0073 |
| STIM1 | Q13586 | Stromal interaction molecule 1 | 0.0081 |
| CSE1L | P55060 | CSE1 chromosome segregation 1-like | 0.0093 |

TABLE 4
Differentially targeted autoantigens in BPH

| Swiss-prot | |
|---|---|
| accession no. | Antigen Description |
| P28223 | 5-hydroxytryptamine (serotonin) receptor 2A |
| P09874 | ADP-ribosyltransferase (NAD+; poly (ADP-ribose) polymerase) |
| P50995 | annexin A11 |
| Q9NYM9 | blocked early in transport 1 homolog (S. cerevisiae) like |
| P51813 | BMX non-receptor tyrosine kinase |
| P35221 | catenin (cadherin-associated protein), alpha 1, 102 kDa |
| O15111 | conserved helix-loop-helix ubiquitous kinase |
| P20248 | cyclin A1 |
| Q9H4B4 | cytokine-inducible kinase |
| P43146 | deleted in colorectal carcinoma |
| Q05193 | dynamin 1 |
| P55010 | eukaryotic translation initiation factor 5 |
| P02751 | fibronectin 1 |
| P06396 | gelsolin (amyloidosis, Finnish type) |
| P04901 | guanine nucleotide binding protein (G protein), beta polypeptide 1 |
| P25388 | guanine nucleotide binding protein (G protein), beta polypeptide 2-like 1 |
| P14317 | hematopoietic cell-specific Lyn substrate 1 |
| P05556 | integrin, beta 1 (fibronectin receptor, beta polypeptide, antigen CD29 includes |
| | MDF2, MSK12) |
| Q13476 | kinase suppressor of ras |
| P52732 | kinesin family member 11 |
| O15066 | kinesin family member 3B |
| O75112 | LIM domain binding 3 |
| O15264 | mitogen-activated protein kinase 13 |
| P49185 | mitogen-activated protein kinase 8 |
| O75970 | multiple PDZ domain protein |
| P43246 | mutS homolog 2, colon cancer, nonpolyposis type 1 (E. coli) |
| P55196 | myeloid/lymphoid or mixed-lineage leukemia (trithorax homolog, Drosophila); |
| | translocated to, 4 |
| P48681 | nestin |
| P15531 | non-metastatic cells 1, protein (NM23A) expressed in |
| Q15233 | non-POU domain containing, octamer-binding |
| Q14980 | nuclear mitotic apparatus protein 1 |
| Q02548 | paired box gene 5 (B-cell lineage specific activator protein) |
| P49023 | paxillin |
| P40855 | peroxisomal farnesylated protein |
| O00633 | phosphatase and tensin homolog (mutated in multiple advanced cancers 1) |
| P41236 | protein phosphatase 1, regulatory (inhibitor) subunit 2 |
| Q99638 | RAD9 homolog (S. pombe) |
| O75759 | RAN binding protein 3 |
| P20936 | RAS p21 protein activator (GTPase activating protein) 1 |
| Q15418 | ribosomal protein S6 kinase, 90 kDa, polypeptide 1 |
| P40763 | signal transducer and activator of transcription 3 (acute-phase response factor) |
| Q99700 | spinocerebellar ataxia 2 (olivopontocerebellar ataxia 2, autosomal dominant, ataxin |
| | 2) |
| Q16637 | survival of motor neuron 1, telomeric |
| O14893 | survival of motor neuron protein interacting protein 1 |
| P70281 | synaptonemal complex protein 3 |
| O43396 | thioredoxin-like, 32 kDa |
| P11387 | topoisomerase (DNA) I |
| Q02880 | topoisomerase (DNA) II beta 180 kDa |
| O14776 | transcription elongation regulator 1 (CA150) |
| Q13885 | tubulin, beta polypeptide |
| P07947 | v-yes-1 Yamaguchi sarcoma viral oncogene homolog 1 |
| P13010 | X-ray repair complementing defective repair in Chinese hamster cells 5 (double- |
| | strand-break rejoining; Ku autoantigen, 80 kDa) |

All references cited herein are fully incorporated by reference. Having now fully described the invention, it will be understood by those of skill in the art that the invention may be practiced within a wide and equivalent range of conditions, parameters and the like, without affecting the spirit or scope of the invention or any embodiment thereof.

1-38. (canceled)

39. A method of diagnostically evaluating a subject for prostate cancer, comprising:
a) obtaining a test biological sample from a said subject;
b) assaying said test biological sample for one or more tumor associated markers (TAMs), wherein said one or more TAMs are selected from a first group consisting of: SATB1 (Q1826); PEX1 (043933); CRP2 (P52943); PSME3 (Q12920); GFAP (P14136); STX6 (043752); SOS1 (Q07889); HSF4 (Q9ULV5); SRP54 (P13624); NHE-3 P19634); PKP2 (Q99960); GRIN2B (Q13224); GSPT2 (PO₆₄₉₃); STAT2 (P52630); and STIM1 (Q13586);
c) comparing the results obtained in step b) with an assay of said one or more TAMs in a control sample; and
d) concluding that said subject is at increased risk of having prostate cancer if the amount of said one or more TAMs in said test biological sample is higher than in said control sample.

40. The method of claim 39, wherein said test biological sample is selected from the group consisting of: blood; plasma; serum; prostate fluid; prostate tissue; and urine.

41. The method of claim 39, wherein the assay of said one or more TAMs is selected from the group consisting of an: ELISA; radioimmunoassay; radioreceptor assay; and antibody profiling assay.

42. The method of claim 39, wherein, in addition to said one or more TAMs from said first group, said test biological sample is assayed for at least one additional TAM selected from the group consisting of: CHD-3 (Swiss protein accession no. Q12873); NFAT (Q13469); CALD1 (Q05682); p53 (P04637); SP11 (P17947); EPHX1 (P07099); DGKq (P52824); TP73 (015350); CSE1L (P55060); EGFR (P00533); AR (P10275); (CCND1); and CASP-8 (Q14790).

43. The method of claim 42, wherein at least 5 different TAMs are assayed.

44. The method of claim 43, wherein said 5 TAMs are: NFAT1 (Q13469), HSF4 (Q9ULV5), p53 (P04637), CASP8 (Q14790), and SP11 (P17947).

45. The method of claim 39, wherein said test biological sample is also assayed for prostate specific antigen.

46. A glass or plastic plate or slide comprising at least 5 different TAMs, wherein:
a) each TAM is attached to a different site on said plate or slide;
b) at least one TAM is selected from the group consisting of: SATB1 (Q1826); PEX1 (O43933); CRP2 (P52943); PSME3 (Q12920); GFAP (P14136); STX6 (O43752); SOS1 (Q07889); HSF4 (Q9ULV5); SRP54 (P13624); NHE-3 P19634); PKP2 (Q99960); GRIN2B (Q13224); GSPT2 (P06493); STAT2 (P52630); and STIM1 (Q13586); and
c) the other TAMs are selected from the group consisting of: SATB1 (Q1826); PEX1 (O43933); CRP2 (P52943); PSME3 (Q12920); GFAP (P14136); STX6 (O43752); SOS1 (Q07889); HSF4 (Q9ULV5); SRP54 (P13624); NHE-3 P19634); PKP2 (Q99960); GRIN2B (Q13224); GSPT2 (P06493); STAT2 (P52630); and STIM1 (Q13586); CHD-3 (Swiss protein accession no. Q12873); NFAT (Q13469); CALD1 (Q05682); p53 (P04637); SP11 (P17947); EPHX1 (P07099); DGKq (P52824); TP73 (015350); CSE1L (P55060); EGFR (P00533); AR (P10275); (CCND1); and CASP-8 (Q14790).

47. The glass or plastic plate or slide of claim 46, wherein each TAM is attached to said plate or slide by a monoclonal antibody that specifically recognizes said TAM.

48. The glass or plastic plate or slide of claim 47, wherein at least 10 TAMs are attached to said plate or slide.

49. An assay for comparing the antibodies present in a sample of blood plasma or serum, comprising:
a) obtaining an immobilized array of TAMs, wherein each TAM is attached to the surface of a solid support by a monoclonal antibody that specifically recognizes said TAM and said TAMs are selected from the group consisting of: EGFR (P00533); AR (P10275); (CCND1); and CASP-8 (Q14790); SATB1 (Q1826); PEX1 (O43933); CRP2 (P52943); PSME3 (Q12920); GFAP (P14136); STX6 (O43752); SOS1 (Qo7889); HSF4 (Q9ULV5); SRP54 (P13624); NHE-3 P19634); PKP2 (Q99960); GRIN2B (Q13224); GSPT2 (P06493); STAT2 (P52630); and STIM1 (Q13586);
b) obtaining test antibodies from a first sample of blood, serum or plasma, and attaching said test antibodies to a first detectable label;
c) obtaining control antibodies from a second sample of blood, serum or plasma and attaching said control antibodies to a second detectable label, wherein said second detectable label can be distinguished from said first detectable label after incubation with said array of immobilized TAMs;
d) incubating said labeled test antibodies and said labeled control antibodies with said array of immobilized TAMs;
e) after the incubation of step d), removing unbound labeled antibodies from said array of immobilized TAMs; and
f) measuring the first and second detectable labels associated with each TAM.

50. The assay of claim 49, wherein said first and said second detectable labels are dyes or fluorescent labels and wherein said first detectable label absorbs or fluoresces at a different wavelength than said second detectable label.

51. The assay of claim 49, wherein said solid support is a glass or plastic plate or slide, and said first and second detectable labels are Cy3 and Cy5 fluorescent dyes.

52. A method of diagnostically evaluating a subject for benign prostate hyperplasia (BPH), comprising:
a) obtaining a test biological sample from said subject;
b) assaying said test biological sample for one or more BPH associated markers (BPHMs), wherein said one or more BPHMs are selected from the group consisting of: 5-hydroxytryptamine (serotonin) receptor 2A (P28223); ADP-ribosyltransferase (NAD+; poly (ADP-ribose) polymerase) (P09874); annexin A11 (P50995); blocked early in transport 1 homolog (S. cerevisiae) like (Q9NYM9); BMX non-receptor tyrosine kinase (P51813); catenin (cadherin-associated protein), alpha 1, 102 kDa (P35221); conserved helix-loop-helix ubiquitous kinase (O15111); cyclin A1 (P20248); cytokine-inducible kinase (Q9H4B4); deleted in colorectal carcinoma (P43146); dynamin 1 (Q05193); eukaryotic translation initiation factor 5 (P55010); fibronectin 1 (P02751); gelsolin (amyloidosis, Finnish type) (P06396); guanine nucleotide binding protein (G protein), beta polypeptide 1 (P04901); guanine nucleotide binding protein (G protein), beta polypeptide 2-like 1 (P25388); hematopoietic cell-specific Lyn substrate 1 (P14317); integrin, beta 1 (fibronectin receptor, beta polypeptide, antigen CD29 includes MDF2, MSK12) (P05556); kinase suppressor of ras (Q13476); kinesin family member 11 (P52732); kinesin family member 3B (O15066); LIM domain binding 3 (O75112); mitogen-activated protein kinase 13 (O15264); mitogen-activated protein kinase 8 (P49185); multiple PDZ domain protein (O75970); mutS homolog 2, colon cancer, nonpolyposis type 1 (E. coli) (P43246); myeloid/lymphoid or mixed-lineage leukemia (trithorax homolog, Drosophila) translocated to, 4 (P55196); nestin (P48681); non-metastatic cells 1, protein (NM23A) expressed in (P15531); non-POU domain containing, octamer-binding (Q15233); nuclear mitotic apparatus protein 1 (Q14980); paired box gene 5 (B-cell lineage specific activator protein) (Q02548); paxillin (P49023); peroxisomal farnesylated protein (P40855); phosphatase and tensin homolog (mutated in multiple advanced cancers 1) (O00633); protein phosphatase 1, regulatory (inhibitor) subunit 2 (P41236); RAD9 homolog (S. pombe) (Q99638); RAN binding protein 3 (O75759); RAS p21 protein activator (GTPase activating protein) 1 (P20936); ribosomal protein S6 kinase, 90 kDa, polypeptide 1 (Q15418); signal transducer and activator of transcription 3 (acute-phase response factor) (P40763); spinocerebellar ataxia 2 (olivopontocerebellar ataxia 2, autosomal dominant, ataxin 2) (Q99700); survival of motor neuron 1, telomeric (Q16637); survival of motor neuron protein interacting protein 1 (O14893); synaptonemal complex protein 3 (P70281); thioredoxin-like, 32 kDa (O43396); topoisomerase (DNA) I (P111387); topoisomerase (DNA) II beta 180 kDa (Q02880); transcription elongation regulator 1 (CA150) (O14776); tubulin, beta polypeptide (Q13885); v-yes-1 Yamaguchi sarcoma viral oncogene homolog 1 (P07947); X-ray repair complementing defective repair in Chinese hamster cells 5 (double-strand-break rejoining; Ku autoantigen, 80 kDa) (P13010);
c) comparing the results obtained in step b) with an assay of said one or more BPHMs in a control sample; and
d) concluding that said subject is at increased risk of having prostate cancer if the amount of said one or more BPHMs in said test biological sample is higher than in said control sample.

53. The method of claim 52, wherein said test biological sample is selected from the group consisting of: blood; plasma; serum; prostate fluid; prostate tissue; and urine.

54. The method of claim 52, wherein the assay of said one or more BPHMs is selected from the group consisting of an: ELISA; radioimmunoassay; radioreceptor assay; and antibody profiling assay.

55. The method of claim 52, wherein at least 5 different BPHMs are assayed.

56. A glass or plastic plate or slide comprising at least 5 different BPHMs, wherein:
a) each BPHM is attached to a different site on said plate or slide;
b) said BPHMs are selected from the group consisting of: 5-hydroxytryptamine (serotonin) receptor 2A (P28223); ADP-ribosyltransferase (NAD+; poly (ADP-ribose) polymerase) (P09874); annexin A11 (P50995); blocked early in transport 1 homolog (S. cerevisiae) like (Q9NYM9); BMX non-receptor tyrosine kinase (P51813); catenin (cadherin-associated protein), alpha 1, 102 kDa (P35221); conserved helix-loop-helix ubiquitous kinase (O15111); cyclin A1 (P20248); cytokine-inducible kinase (Q9H4B4); deleted in colorectal carcinoma (P43146); dynamin 1 (Q05193); eukaryotic translation initiation factor 5 (P55010); fibronectin 1 (P02751); gelsolin (amyloidosis, Finnish type) (P06396); guanine nucleotide binding protein (G protein), beta polypeptide 1 (P04901); guanine nucleotide binding protein (G protein), beta polypeptide 2-like 1 (P25388); hematopoietic cell-specific Lyn substrate 1 (P14317); integrin, beta 1 (fibronectin receptor, beta polypeptide, antigen CD29 includes MDF2, MSK12) (P05556); kinase suppressor of ras (Q13476); kinesin family member 11 (P52732); kinesin family member 3B (O15066); LIM domain binding 3 (O75112); mitogen-activated protein kinase 13 (O15264); mitogen-activated protein kinase 8 (P49185); multiple PDZ domain protein (O75970); mutS homolog 2, colon cancer, nonpolyposis type 1 (E. coli) (P43246); myeloid/lymphoid or mixed-lineage leukemia (trithorax homolog, Drosophila) translocated to, 4 (P55196); nestin (P48681); non-metastatic cells 1, protein (NM23A) expressed in (P15531); non-POU domain containing, octamer-binding (Q15233); nuclear mitotic apparatus protein 1 (Q14980); paired box gene 5 (B-cell lineage specific activator protein) (Q02548); paxillin (P49023); peroxisomal farnesylated protein (P40855); phosphatase and tensin homolog (mutated in multiple advanced cancers 1) (O00633); protein phosphatase 1, regulatory (inhibitor) subunit 2 (P41236); RAD9 homolog (S. pombe) (Q99638); RAN binding protein 3 (O75759); RAS p21 protein activator (GTPase activating protein) 1 (P20936); ribosomal protein S6 kinase, 90 kDa, polypeptide 1 (Q15418); signal transducer and activator of transcription 3 (acute-phase response factor) (P40763); spinocerebellar ataxia 2 (olivopontocerebellar ataxia 2, autosomal dominant, ataxin 2) (Q99700); survival of motor neuron 1, telomeric (Q16637); survival of motor neuron protein interacting protein 1 (014893); synaptonemal complex protein 3 (P70281); thioredoxin-like, 32 kDa (043396); topoisomerase (DNA) I (P11387); topoisomerase (DNA) II beta 180 kDa (Q02880); transcription elongation regulator 1 (CA150) (O14776); tubulin, beta polypeptide (Q13885); v-yes-1 Yamaguchi sarcoma viral oncogene homolog 1 (P07947); X-ray repair complementing defective repair in Chinese hamster cells 5 (double-strand-break rejoining; Ku autoantigen, 80 kDa) (P13010).

57. The glass or plastic plate or slide of claim 56, wherein each BPHM is attached to said plate or slide by a monoclonal antibody that specifically recognizes said BPHM.

58. The glass or plastic plate or slide of claim 57, wherein at least 10 TAMs are attached to said plate or slide.
